(19) United States
(12) Patent Application Publication
(10) Pub. No.: US 2025/0041081 A1
(43) Pub. Date: Feb. 6, 2025

(54) JOINT AND METHOD FOR SETTING A STARTING POSITION

(71) Applicant: OTTOBOCK SE & CO. KGAA, Duderstadt (DE)

(72) Inventors: Martin PUSCH, Duderstadt (DE); Johannes FLOORS, Duderstadt (DE); Sven KALTENBORN, Duderstadt (DE)

(21) Appl. No.: 18/716,684

(22) PCT Filed: Dec. 7, 2022
(86) PCT No.: PCT/EP2022/084857

§ 371 Date: Jun. 5, 2024

(30) Foreign Application Priority Data
Dec. 7, 2021 (DE) 10 2021 132 201.4

(51) Int. Cl. A61F 2/74; A61F 2/50; A61F 2/66

(52) U.S. Cl. CPC A61F 2/748; A61F 2/6607; A61F 2002/5006; A61F 2002/5016

(57) ABSTRACT

The invention relates to a joint for an orthopedic device. The joint has a first joint part (38), a second joint part (34), which is pivotally arranged on the first joint part (38) about a pivot axis (36), and a hydraulic system with—a first hydraulic chamber (8),—a second hydraulic chamber (10), which is connected to the first hydraulic chamber (8) by means of at least one fluidic connection (12), and—at least one valve (14), which is designed to open and close the fluidic connection (12) and which is arranged and designed such that hydraulic fluid flows from the first hydraulic chamber (8) into the second hydraulic chamber (10) or vice versa when the first joint part (38) is pivoted relative to the second joint part (34), wherein the hydraulic system has at least one volume (18), which has a first sub-volume and a second sub-volume, and at least one additional fluid line (16), which has a first sub-line (24) and a second sub-line (26). The first sub-volume is connected to the first hydraulic chamber (8) via the first sub-line (24), and the second sub-volume is connected to the second hydraulic chamber (10) via the second sub-line (26). The first sub-volume is separated from the second sub-volume by a movable separating device.

The invention relates to a joint for an orthopedic device, the joint comprising a first joint part and a second joint part, which is arranged on the first joint part such that it can be swivelled about a swivel axis. The swivel axis may be a fixed swivel axis, which extends along a shaft for example, or a virtual swivel axis. The joint also has a hydraulic system with a first hydraulic chamber, a second hydraulic chamber that is fluidically connected to the first hydraulic chamber by at least one fluid connection, and at least one valve that is configured to open and close the fluid connection. The hydraulic system is arranged and configured such that hydraulic fluid flows from the first hydraulic chamber into the second hydraulic chamber or vice versa when the first joint part is swivelled relative to the second joint part. The invention also relates to a method for setting a starting position of the first joint part relative to the second joint part of such a joint and an orthopedic device that has such a joint.

Within the context of the present invention, an orthopedic device refers in particular to a prosthesis or an orthosis. They are preferably designed for the lower limb and the joint is used as a knee joint, a hip joint or an ankle joint.

Joints according to the preamble, which are referred to as hydraulic joints, have been known from the prior art for many years and can essentially be used in two different ways.

It is particularly, but not exclusively, advantageous for ankle joints if a position of the first joint part relative to the second joint part can be adjusted and locked in different positions. This renders it possible, for example, to freely adjust an angle between a lower leg section and a foot section of a prosthesis, which in this case forms the first and second joint part. For example, if the wearer of such a prosthesis changes their footwear, it generally also leads to a change in heel height; said adjustability allows this to be taken into account. When using the prosthesis, it is not intended that hydraulic fluid is transferred from one hydraulic chamber to another hydraulic chamber after adjusting the heel height. Such a joint, which does not allow any movement during operation, is highly reliable, thereby giving the wearer of a prosthesis or orthosis equipped with such a joint a sense of security.

Alternative embodiments of hydraulic joints for orthopedic devices provide that the fluid connection is not closed when the joint is in operation. A movement of the two joint parts relative to each other is possible as a result. However, depending on the flow resistance generated by the fluid connection, the movement is damped to a greater or lesser extent. Preferably, it is possible to render the flow resistance and therefore also the damping of the movement adjustable, for example by way of a throttle valve. The prior art includes valve arrangement designs which feature combinations of throttle valve and non-return valves, so that the flow resistances that counter a flow of hydraulic fluid from one hydraulic chamber into another hydraulic chamber can be adjusted individually and preferably differently for different flow directions. Such a joint, with which a damped movement is possible, enables a very natural gait pattern on the one hand and a high level of comfort on the other, as mechanical impacts can be absorbed.

However, it is disadvantageous that a combination of the two effects, of a fixed joint angle on the one hand and a damped movement on the other, seems impossible without a mechatronic control system.

U.S. Pat. No. 9,132,023 B2 discloses an artificial ankle joint that enables a heel-height adjustment on the one hand and dynamic damping on the other. To this end, it has four hydraulic chambers that form two pairs connected to one another. If the joint is to be dynamically damped, the connection between the two chambers of the first pair is closed. The two other chambers are then fluidically connected, so that a movement of the joint displaces hydraulic fluid from one chamber into the other. Damping can then be adjusted via the flow resistance. Conversely, if a heel height is to be adjusted, the closed connection between the chambers of the first pair must be opened and the open connection between the chambers of the second pair closed or it must be ensured in another way that no hydraulic fluid can be exchanged between the chambers of the second pair. The heel height of the first pair can then be adjusted by changing the ratio of the size of the chambers of the first pair. However, it is disadvantageous that the size ratio of the chambers of the second pair is not reproducible and therefore the angle in the ankle joint at which the heel height is set cannot be adjusted reproducibly. Moreover, the construction requires a relatively large installation space and is heavy due to the many high-mass elements. This is particularly disadvantageous for ankle joints as they are worn away from the body and have to be accelerated considerably when walking, for example. This causes significant moments of inertia that have to be overcome.

The invention therefore aims to eliminate or at least reduce this disadvantage.

The invention solves the addressed task by way of a joint for an orthopedic device according to the preamble of claim 1, which is characterized in that the hydraulic system has at least one volume, which comprises a first partial volume and a second partial volume, and at least one further fluid line, which comprises a first partial line and a second partial line, wherein the first partial volume is connected to the first hydraulic chamber via the first partial line and the second partial volume is connected to the second hydraulic chamber via the second partial line and that the first partial volume is separated from the second partial volume by a displaceable separating device.

Even if the first partial line and the second partial line are jointly referred to as a further fluid line, this does not mean that they are fluidically connected. Rather, the volume with the first partial volume and the second partial volume, the latter two being separated by the displaceable separating device, is situated between the two partial lines. If fluid is directed out of the first hydraulic chamber, through the first partial line and into the first partial volume, the displaceable separating device must be displaced so as to enlarge the first partial volume. This inevitably leads to a reduction in the second partial volume, so that fluid is directed out of the second partial volume, through the second partial line and into the second hydraulic chamber.

The separating device must therefore be able to change the size of the abutting partial volumes. This can be achieved, for example, by way of a displaceable piston, also referred to as a flying piston. Alternatively or additionally, the separating device has a membrane, which is preferably stretched across the cross-section of the volume. If fluid is now directed into one of the two partial volumes, the membrane bulges due to the increased pressure on the one side, thereby increasing the first partial volume at the expense of the second partial volume, which decreases in the process.

Therefore, even if the fluid connection between the two hydraulic chambers is closed by the valve, the separating device can be moved within the volume in the other fluid line. It is advantageous if the separating device can be moved within the volume, which may be a cylinder for example, but fluid itself cannot pass through the separating device. The separating device preferably rests against the inner wall of the volume. The separating device and volume are preferably designed as a cylinder and a piston adapted to it and can be designed as a longitudinally displaceable system or a rotary hydraulic system. A bent or curved piston is also possible. It is important that a displacement of the piston causes fluid to be displaced from the first hydraulic chamber into the second hydraulic chamber or vice versa. It is therefore sufficient for the further fluid line to comprise a first partial line, which connects the first hydraulic chamber with a first partial volume where the separating device is located, and a second partial line, which connects the second hydraulic chamber with a second partial volume. In this case, the first partial volume and the second partial volume are separated from each other by the separating device.

When the fluid line between the two hydraulic chambers is closed, fluid can be exchanged between the first hydraulic chamber and the first partial volume through the first partial line. It is likewise possible to exchange hydraulic fluid between the second hydraulic chamber and the second partial volume through the second partial line. Since the overall volume, composed of the first partial volume and the second partial volume, remains constant, even when the separating device is moved, a quantity of fluid directed into one of the two partial volumes must be removed from the respective other partial volume.

Preferably, the volume in which the separating device, for example the movably mounted piston, is smaller than the volume of the first hydraulic chamber and smaller than the volume of the second hydraulic chamber. The range of movement of the joint that is also possible when the fluid connection is closed, i.e. when the first partial line and/or the second partial line is closed, between the first hydraulic chamber and the second hydraulic chamber is then small in relation to the range of movement of the joint when the fluid connection is open. If the fluid connection between the first hydraulic chamber and the second hydraulic chamber is closed, fluid can only be moved out of one of the two hydraulic chambers into the volume in the fluid line, where it causes the separating device, for example the moveably mounted piston, to be displaced. On the opposite side of the separating device, this causes fluid to be directed from the volume into the respective other hydraulic chamber. As soon as the separating device can move no further in this direction, the joint can also be moved no further in this direction, unless there is another volume available for the hydraulic fluid, for example in the form of an equalizing volume.

In one preferred embodiment, this range of movement is restricted by the separating device, such as the piston, within the volume striking an end stop on at least one side, preferably on two sides, when a certain position is reached. From this point onwards, further movement in this direction of movement is no longer possible and the range of movement in this direction of movement restricted. Preferably, at least one of the end stops, particularly preferably both end stops, is equipped with a spring or a damping element, by means of which a movement is possible in a limited range when a sufficiently large force is applied, even if the end stop has already been reached.

The two hydraulic chambers are preferably connected to each other via at least two fluid connections. Valve arrangements are preferably arranged in the two fluid connections, each of the former having a non-return valve, wherein the two non-return valves act in different flow directions. As a result, the flow resistances for different directions can be selected independently from each other. Both fluid connections can preferably be sealed by a valve.

This configuration is advantageous for knee joints, for example. When the valves of the fluid connections are open, a normal flexion and extension of the joint is possible and hydraulic fluid is directed in each case from one hydraulic chamber into the respective other hydraulic chamber via one of the two fluid connections. The flexion resistance and the extension resistance can be adjusted independently of one another via the valve arrangement in the respective fluid connection. When the two valves are closed, a movement of the two joint parts relative to one another is still possible as fluid can be directed from the hydraulic chambers into the partial volumes and vice versa. This enables, for example, a flexing of the knee in the stance phase.

The at least first partial line and/or the second partial line preferably has at least one throttle, by means of which a flow resistance through the first line and/or the second partial line can be adjusted. It is understood that a throttle in the first partial line also changes the flow resistance of the first partial line and a throttle in the second partial line changes the flow resistance of the second partial line. The throttle, which can be designed as a throttle valve for example, is arranged in the first partial line or the second partial line. Of course, it is also possible to use more than one throttle, of which preferably at least one is arranged in the first partial line and at least one in the second partial line.

Advantageously, at least one valve arrangement is arranged in the first partial line and/or the second partial line, by means of which a flow resistance through the first line and/or the second partial line can be adjusted differently for different flow directions. Such valve arrangements are known in principle from the prior art. They have combinations of throttle valve and non-return valve, which are arranged to act in parallel. The non-return valve ensures that the throttle valve is only passed through in a single flow direction, namely the direction in which the non-return valve prevents flow, and the throttle valve adjusts the desired flow resistance. In one preferred embodiment, the at least one fluid line has two such combinations, wherein the two non-return valves act in opposing directions. The one combination can therefore only be passed through in the first flow direction and the other combination only in the second flow direction.

Preferably, the separating device can be displaced in at least one direction, particularly preferably in two opposite directions, in each case against a spring force applied by a spring element. This also changes the resistance to a displacement of the separating device within the volume and thus also a displacement of the fluid.

Preferably, the movement of the separating device in at least one direction, especially preferably in two opposite directions, is limited is each case by an end stop, which preferably comprises a damping element. The damping element is preferably an elastomer block or a disk spring. Preferably at least one of the end stops, but particularly preferably both end stops, is designed to be adjustable so that a range of movement of the separating device can be adjusted.

In one preferred embodiment, the first hydraulic chamber is separated from the second hydraulic chamber by a main piston, which is arranged and configured such that it can be moved by swivelling the first joint part relative to the second joint part. The term “main piston” serves only to distinguish it from the piston in the volume of the fluid line and does not imply any size or mass ratios. The use of a single main piston enables an especially simple structural design. The first hydraulic chamber and the second hydraulic chamber can be arranged in the same cylinder and in this case are separated from each other by the main piston. The main piston can also be designed to be longitudinally displaceable or in the form of a rotary hydraulic system, in which it performs a rotary movement when displaced.

The first partial line and/or second partial line preferably extend(s) through the main piston. Particularly preferably, the volume in the fluid line, in which the separating device is located, is arranged within the main piston. The entire fluid line is preferably located within the main piston. This results in an increased engineering effort, but reduces the installation space required, which is generally limited in joints for orthopedic devices.

The first hydraulic chamber and the second partial volume are preferably arranged in a common cylinder. Alternatively or additionally, the second hydraulic chamber and the first partial volume are preferably arranged in a common cylinder.

Preferably, the first hydraulic chamber and the second partial volume are separated from each other by a first piston. Alternatively or additionally, the second hydraulic chamber is separated from the first partial volume by a second piston.

Especially preferred embodiments include a cylinder in which one of the two hydraulic chambers and the corresponding partial volume are positioned, which are separated by the respective piston. The volume of the respective hydraulic chamber is preferably delimited by the wall of the cylinder and the piston. In addition, the respective hydraulic chamber is delimited by the main piston. The main piston and the first or second piston preferably delimit the hydraulic chamber on opposite sides. Preferably, various movements of the individual components relative to each other are possible, which have different effects on the hydraulic system and thus on the position of the two joint parts.

When the fluid connection between the two hydraulic chambers is closed, fluid can no longer move from one hydraulic chamber into the other hydraulic chamber. However, the partial lines render it possible to direct fluid from one hydraulic chamber into the corresponding partial volume. As a result, fluid can move from the first hydraulic chamber into the first partial volume and from the second hydraulic chamber into the second partial volume. When fluid is directed from the first hydraulic chamber into the first partial volume, and the first partial volume and the second hydraulic chamber are arranged in the same cylinder, the amount of fluid in the cylinder increases, but not the volume of the second hydraulic chamber. In this case, the main piston moves parallel to the second piston as a result. The movement of the main piston also causes a reduction in the volume of the first hydraulic chamber. The volume of the second partial volume remains unchanged. A movement of the two joint parts relative to one another occurs, although the fluid connection between the two hydraulic chambers is closed.

The first partial line and/or the second partial line can preferably be sealed by at least one closing valve. Particularly preferably, both partial lines can be sealed by a joint closing valve. In one preferred embodiment, the at least one closing valve is designed in such a way that it opens when the valve, by means of which the fluid connection between the two hydraulic chambers can be sealed, is closed and vice versa. In this case, vice versa means that the at least one closing valve is closed when the valve, by means of which the fluid connection between the two hydraulic chambers can be sealed, is opened. The valve and the at least one closing valve preferably form a single common valve.

The invention also solves the addressed task by way of a method for setting a starting position of the first joint part relative to the second joint part of a joint of the type described here, the method comprising the following steps:
positioning the separating device in a predetermined rest position,
opening the fluid connection by activating the valve,
swivelling the first joint part relative to the second joint part until the starting position is reached, and
closing the fluid connection.

Given that the predetermined rest position of the separating device, for example the moveably mounted piston, is assumed before any setting of the starting position, the starting position is easily reproduced. To this end, the separating device, i.e. the piston in the present example, need only be brought into its rest position. For example, if the joint is an ankle joint, the starting position corresponds to a heel height.

The starting position preferably corresponds to a predetermined joint angle between the first joint part and the second joint part.

Preferably, the separating device is displaced within the volume up to an end stop in order to position the separating device in the rest position. To this end, a torque acting about the swivel axis is preferably applied to the first joint part and/or the second joint part. If the predetermined rest position of the separating device is at an end stop, it is especially easy to reach and also easy to adjust for the user of the orthopedic device, such as a prosthesis. The user need only apply a corresponding torque. For example, if the joint is used as an ankle joint of a lower leg prosthesis between a lower leg section and a foot section, the user can, for example, put weight on the forefoot, thereby applying a corresponding torque, causing the separating device to be displaced into its predetermined rest position. Such a torque can also be applied manually. The disadvantage, however, is that the separating device can then only be moved in one direction after setting the starting position, namely away from the end stop.

Alternatively, it is therefore advantageous not to apply a torque acting about the swivel axis to the first joint part and/or the second joint part in order to position the piston in the rest position. To move the separating device in this situation into its predetermined rest position, at least one force must be applied to it that moves it into said rest position. This can be done, for example, by arranging one or more spring elements within the volume in which the piston is movably mounted, which each apply a spring force to it. In a situation in which no external torque and no external forces are acting, said spring elements ensure that the separating device, such as the moveable piston, is brought into its rest position. It is then generally not at an end stop, so that a movement in both directions is possible after setting the starting position.

The spring elements are preferably designed and configured in such a way that they overcome forces and torques exerted and caused by the force of gravity and move the joint into the neutral position, in which the separating device, such as the moveable piston, is in the rest position.

It may be advantageous to use one or multiple spring elements, the spring force of which is enough to move the separating device to one of its end stops and thus reach the rest position.

The invention also solves the addressed task by way of an orthopedic device with a joint of the type described here, which is characterized in that the joint is a hip joint, an ankle joint or a knee joint.

In the following, embodiment examples of the invention will be explained in more detail with the aid of the accompanying drawings. They show:

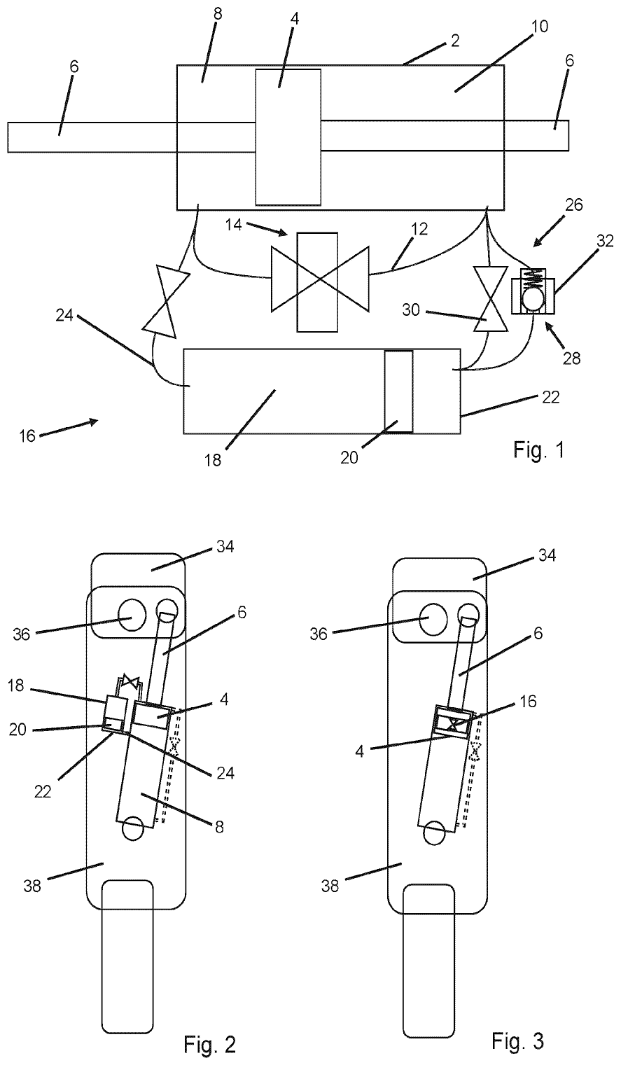

FIG. 1—a schematic circuit diagram of a hydraulic system,

FIGS. 2 to 5—schematic representations of joints according to different embodiment examples of the present invention,

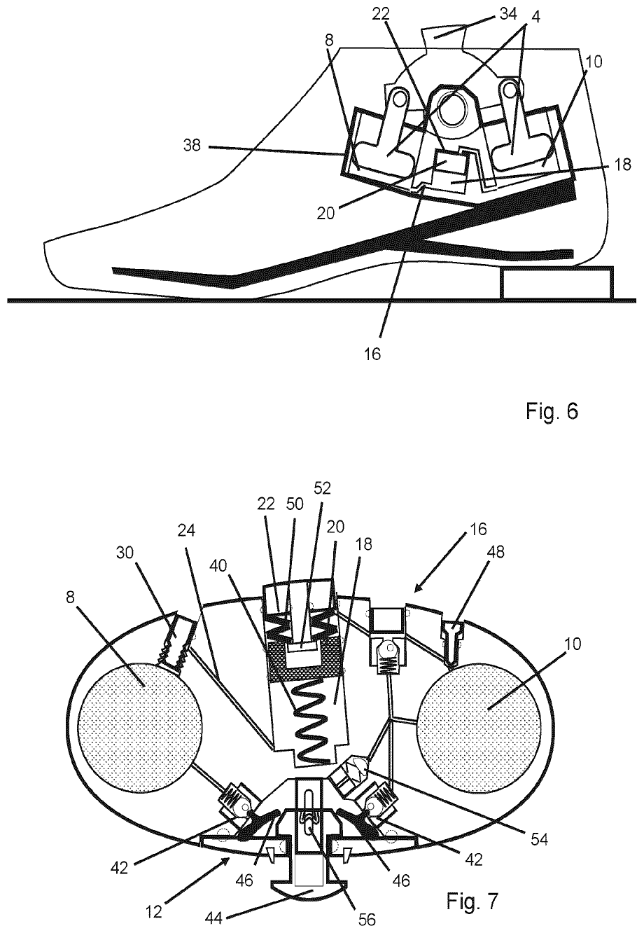

FIG. 6—a schematic representation of a prosthetic foot,

FIG. 7—a schematic top view in a sectional representation,

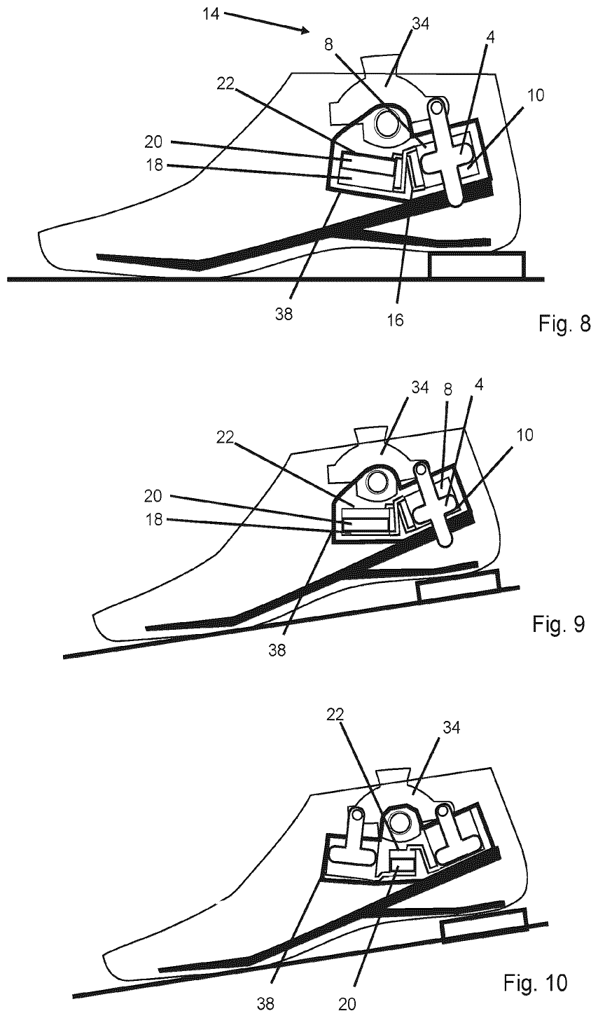

FIGS. 8 and 9—schematic representations of a prosthetic foot according to a further embodiment example of the present invention in two different positions,

FIG. 10—the prosthetic foot from FIG. 6 in a second position,

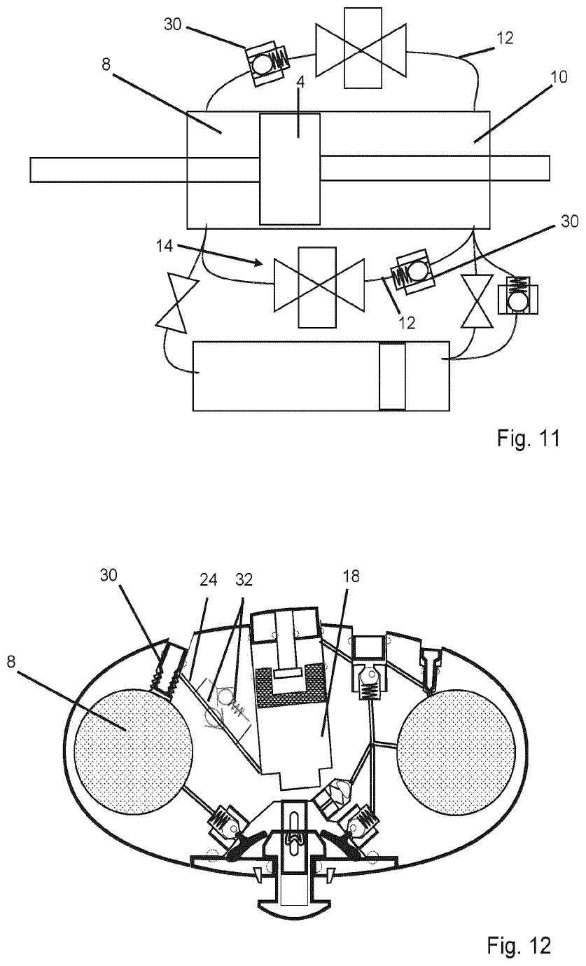

FIG. 11—a different schematic circuit diagram,

FIGS. 12 to 17—top views of schematic sectional representations of various embodiments,

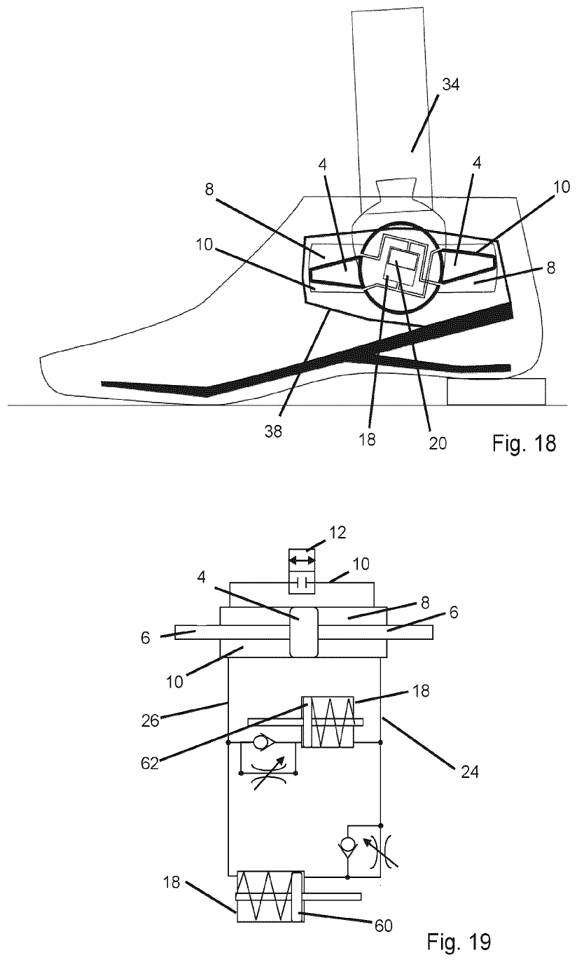

FIG. 18—the schematic representation of a prosthetic foot with a rotary hydraulic system,

FIGS. 19 to 21—schematic circuit diagrams of further hydraulic systems and

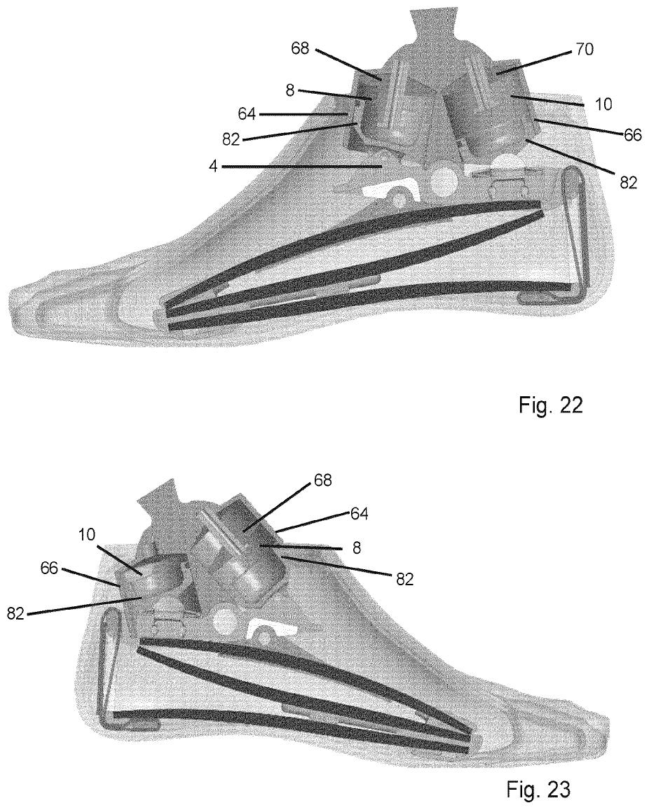

FIGS. 22 and 23—a schematic representation of further prosthetic feet.

FIG. 1 shows the schematic representation of a circuit diagram for a hydraulic system of a joint according to an embodiment example of the present invention. A main piston 4 is arranged in a cylinder 2, wherein the former can be displaced left and right in the representation shown. It is connected to two piston rods 6 that guide its movement. A first hydraulic chamber 8 and a second hydraulic chamber 10, which are separated from each other by the main piston 4, are located in the cylinder 2. A fluid connection 12 connects the first hydraulic chamber 8 to the second hydraulic chamber 10, a valve 14 being located in the fluid connection 12 that can be opened and closed, meaning that the fluid connection 12 can also be opened and closed. If the fluid connection 12 is opened, hydraulic fluid can flow from the first hydraulic chamber 8 into the second hydraulic chamber 10 and vice versa when the main piston 4 moves. The damping of this movement of the main piston 4 can be adjusted via a potentially adjustable flow resistance caused by the valve 14. If the fluid connection 12 is closed, however, the hydraulic fluid cannot flow through the fluid connection 12.

In addition, the hydraulic system has a further fluid line 16. It comprises multiple elements. It has a volume 18 in which a piston 20 is moveably arranged. This piston 20 can also be moved left and right in the embodiment example shown. However, in this embodiment it does not have a piston rod, but is designed as a flying piston. This is advantageous, but not essential. The piston 20 may also be configured with a piston rod. The piston 20 divides the volume 18 into a first part, which is to the left of the piston 20 in the embodiment example shown, and a second part, which is to the right of the piston 20 in the embodiment example shown. The first part of the volume is connected to the first hydraulic chamber 8 via a first partial line 24. The second part of the volume is connected to the second hydraulic chamber 10 via a second partial line 26. A valve arrangement 28 is located in the second partial line, said arrangement comprising a combination of throttle valve 30 and non-return valve 32. This allows a flow resistance against the fluid flowing through the valve arrangement 28 to be adjusted in a flow direction.

FIG. 2 depicts a joint according to an embodiment example of the present invention as part of a schematically illustrated knee prosthesis. The main piston 4 is arranged with its piston rod 6 on the second joint part 34, which is arranged on a first joint part 38 such that it can be swivelled about a swivel axis 36. In the representation shown, the piston 20 is located at the lower end stop 22 and can therefore only be moved in one direction, upwards in FIG. 2. This occurs when the main piston 4 is moved downwards and fluid moves from the first hydraulic chamber 8 through the first partial line 24 and into the volume 18. This enables a flexing of the joint in the stance phase, for example, which makes walking with the prosthesis more gentle for the wearer and the gait pattern more natural.

FIG. 3 shows a similar embodiment. Here, the piston rod 6 is also coupled with the second joint part 34 of the knee joint, which in turn is connected to the first joint part 38 about the swivel axis 36. However, unlike in FIG. 2 the volume 18 and the entire fluid line 16 is now located within the main piston 4, wherein the fluid line 16 is only schematically depicted for the sake of clarity.

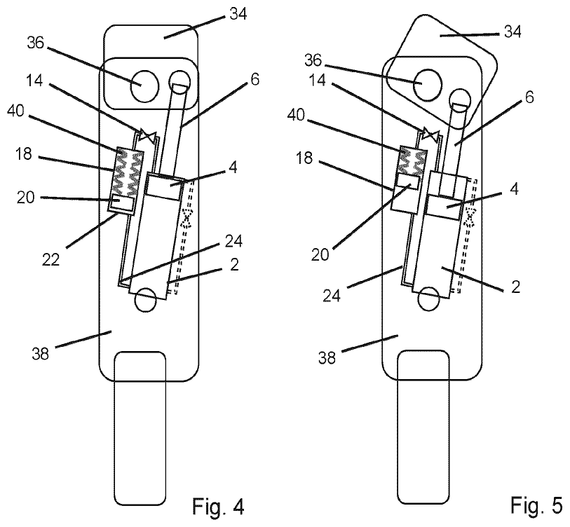

FIGS. 4 and 5 show the identical embodiment. The main piston 4 is in the cylinder 2 and is fixed with the piston rod 6 to the second joint part 34. Unlike in the embodiments in FIGS. 2 and 3, there is a spring element 40 in the volume 18. Said spring element pushes the piston 20 into the rest position at the end stop 22 shown in FIG. 4. Therefore, if the piston 20 is to be moved into its predetermined rest position, it is sufficient to not apply a torque to the first joint part 38 and/or the second joint part 34. The spring element 40 pushes the piston 20 into the rest position. If the second joint part 34 is now swivelled relative to the first joint part 38 about the swivel axis 36, the main piston 4 in the cylinder 2 is moved downwards and pushes fluid through the first partial line 24 into the volume 18, thus moving the piston 20 upwards against the force applied by the spring element 40. This situation is shown in FIG. 5 and denotes flexing in the stance phase.

The connection between the hydraulic chamber and the volume 18 can be closed by way of the valve 14. This renders the movement of the piston 20 impossible.

FIG. 6 depicts a further embodiment example of the present invention as an ankle joint. The first joint part 38 is the prosthetic foot, which is arranged on the second joint part 34 such that it can be swivelled. In the embodiment example shown, the second joint part 34 is configured to be connected to a lower leg element. The piston 4 is designed in the form of two main pistons 4, which form so-called swivel pistons and are each arranged on the second joint part 34 such that they can be swivelled. In each case, the first hydraulic chamber 8 and the second hydraulic chamber 10 are located below the main piston 4. Between the two hydraulic chambers 8, 10 is the fluid line 16, which connects the two hydraulic chambers 8, 10 and in which the volume 18 with the moveable piston 20 is located. In the position depicted in FIG. 6, the moveable piston 20 is positioned at one of its end stops 22, so that a movement of the moveable piston 20 within the volume 18 is only possible in one direction. In FIG. 6 this is a plantar flexion, i.e. a downward movement.

FIG. 7 shows a schematic sectional representation of a top view of the embodiment in FIG. 6. The first hydraulic chamber 8 and the second hydraulic chamber 10 are connected to each other via the fluid connection 12. The valve 14 is designed as a valve arrangement and has two non-return valves 42, each of which can open or close the connection to one of the two hydraulic chambers 8, 10. The arrangement also has a push button 44, which is designed in such a way that, if it is pressed in, i.e. moved upwards in FIG. 7, it actuates the two levers 46 and thus opens the two non-return valves 42. The first partial line 24 is connected to the first hydraulic chamber 8 by a throttle valve 30. A disk spring 50 is depicted at the upper end stop 22, by means of which the end stop 22 is damped. The preload of said disk spring 50 can be adjusted via the adjustable driver 52. The embodiment example shown also depicts a relief valve 54 as well as an opening mechanism 56, by means of which the fluid connection 12 can be opened.

The fluid line 16, composed of multiple partial lines and the volume 18 in the embodiment example shown, is also located between the two hydraulic chambers 8, 10. The piston 20 is located in said volume, the former being pre-tensioned upwards by the spring element 40 in FIG. 7. The spring element 40 is configured to move the piston 20 into its rest position when no other external forces beyond the force of gravity are acting. If the fluid connection 12 is closed, as shown in FIG. 7, a movement of the joint can be achieved by slightly opening the adjustment valve 48. As a result, fluid can flow from the second hydraulic chamber 10 into the volume 18, for example during a heel strike, when increased pressure builds up in the second hydraulic chamber 10, causing the piston 20 to move downwards against the spring force of the spring element 40. A corresponding quantity of fluid flows from the partial volume below the piston 20 into the first hydraulic chamber 8, so that the second joint part 34 moves relative to the first joint part 38.

FIGS. 8 and 9 depict a prosthetic foot similar to the one in FIG. 6. The main difference is that the two hydraulic chambers 8, 10 are separated by a single main piston 4. The two hydraulic chambers 8, 10 are again connected by the fluid line 16 in which the volume 18 with the moveable cylinder 20 is located. As in FIG. 6, the moveable piston 20 is resting against one of its end stops 22 and can therefore only be moved in one direction, downwards in FIG. 8. This position of the moveable piston 30 is preferably assumed when the heel height of the prosthetic foot is determined: in the present embodiment example, essentially the position of the main piston 4 between the hydraulic chambers 8, 10. Once this has happened, the fluid connection 12, which is not shown in FIGS. 6, 8, 9 and 10 and is preferably opened to adjust the heel height, is preferably closed. It is then no longer possible for the fluid to flow from the one hydraulic chamber 8, 10, through the fluid connection 12 and into the respective other hydraulic chamber 10, 8.

FIG. 9 illustrates this situation. Although the fluid connection 12 is closed, the angle between the first joint part 38 and the second joint part 34 has changed compared to the situation in FIG. 8, wherein the main piston 4 has been displaced. As a result, fluid has been displaced from the second hydraulic chamber 10 into the volume 18. In FIG. 9, said fluid is situated above the moveable piston 20 and has moved it downwards. In addition, fluid situated below the moveable piston 20 in FIG. 8 has moved from the volume into the first hydraulic chamber 8.

FIG. 10 shows the situation from FIG. 9 with a prosthetic foot from FIG. 6. The moveable piston 20 moved away from its end stop 22 as the angle between the first joint part 38 and the second joint part 34 changed.

FIG. 11 corresponds to the representation from FIG. 1. The difference, however, is that the first hydraulic chamber 8 and the second hydraulic chamber 10, which are separated from each other by the main piston 4, are no longer connected by one fluid connection 12, but by two fluid connections 12. In both fluid connections 12 there is a valve 14 as well as a throttle valve 30. The valves 14 and/or the throttle valve 30 may be designed differently so as to achieve, for example, different flow resistances for different flow directions of the fluid.

The representations in FIGS. 12 to 17 correspond to the representation in FIG. 7. To avoid repetitions, only the differences shall discussed. Compared to FIG. 7, in FIG. 12 there is a valve arrangement containing two non-return valves 32 in the first partial line 24, which connects the first hydraulic chamber 8 to the volume 18 via the throttle valve 30. Said non-return valves act in different directions, wherein the upper of the two non-return valves 32 in FIG. 12 is spring-loaded. The fluid that flows through this first partial line 24 must pass through the throttle valve 30 regardless of the flow direction.

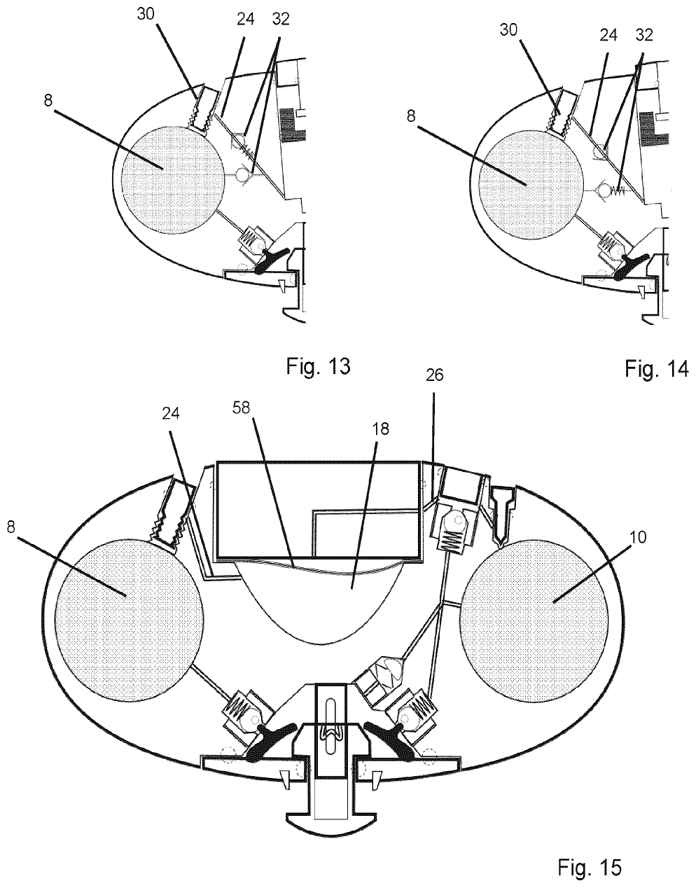

This is different in FIGS. 13 and 14, each of which depicts a section from a corresponding representation. Again, one of the non-return valves 32 is positioned in the first partial line 24. In the embodiment example shown, this is the spring-loaded non-return valve, which allows fluid to flow from the first hydraulic chamber 8, through the throttle valve 30, through the first partial line 24 and into the volume 18 when the pressure is correspondingly high. Fluid cannot pass through this non-return valve 32 in the opposite direction; rather, it passes through the non-spring-loaded non-return valve 32. However, the latter is not arranged in a by-pass in the embodiment example shown, such that the fluid does not have to pass through the throttle valve 30 in this direction.

FIG. 14 illustrates the reversed situation. The non-spring-loaded non-return valve 32, which allows a flow from the volume 18 towards the height of the first hydraulic chamber 8, is positioned in the first partial line 24 in such a way that the fluid flowing through this partial line 24 in this direction passes through the throttle valve 30. The non-return valve 32 acting in the opposite direction, which is spring-loaded, is arranged in the by-pass, so that fluid taking this path does not pass through the throttle valve 30. The skillful selection of the throttle valve and spring of the spring-loaded non-return valve 32 renders it possible to simply and individually adjust the flow resistance for different flow directions.

FIGS. 15 and 17 depict a different embodiment of the present invention. The volume 18 is no longer divided into the two partial volumes by the piston 20, but by a membrane 58. This does not change how it works. Fluid from the first hydraulic chamber 8 can still get into volume 18 below the membrane 58 through the first partial line 24. Fluid from the second hydraulic chamber 10 can get into the second partial volume above the membrane 58 via the second partial line 26. The membrane 58 is designed to be elastic and can thus assume different positions, depending on the prevailing pressure conditions.

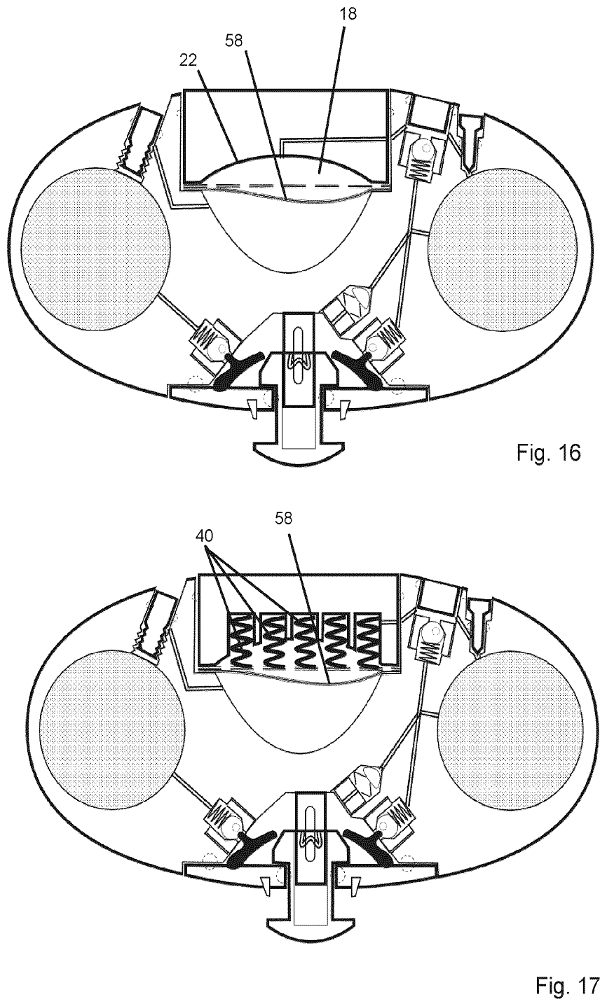

FIGS. 16 and 17 show modified embodiments, each of which however is equipped with the membrane 58. While FIG. 16 only differs from FIG. 15 in that the geometric shape of the volume 18 has been modified, FIG. 17 shows additional spring elements 40. The membrane 58 is preferably designed to be flexible and elastic so that it can rest against the wall delimiting the volume 18 on at least one side. Said wall then acts as an end stop 22 and thus delimits the maximum effective range of the membrane 58. While in this case the end stop 22 in FIG. 16 is designed to be undamped, the embodiment in FIG. 17 is damped by way of the spring elements 40. The membrane 58 initially rests against the lower end of the spring elements 40 in FIG. 17. If further fluid is directed into the first partial volume, shown below the membrane 58 in FIG. 17, the pressure in this area increases, causing the membrane to compress the spring elements, thus enabling a further movement.

FIG. 18 schematically depicts a prosthetic foot with the first joint part 38 and the second joint part 34. The first hydraulic chamber 8 and the second hydraulic chamber 10 are each composed of two parts, which in each case are connected to each other. The prosthetic foot in FIG. 15 has a rotary hydraulic system. The main piston 4 also comprises two parts which are connected to each other in a torque-proof manner. If the joint is moved, the two joint parts 34, 38 are swivelled against each other and the main piston 4 is moved relative to the hydraulic chambers. The parts of the hydraulic chambers 8, 10 upstream of the main piston 4 in the direction of rotation are rendered smaller and the parts of the hydraulic chambers 8, 10 downstream of the main piston 4 in the direction of rotation are enlarged. In FIG. 18, the piston 20 is arranged in the volume 18 between the two parts of the main piston 4 in the area of the axis of rotation of the joint.

FIG. 19 schematically depicts a diagram of a further hydraulic system for a joint for an orthopedic device according to a further embodiment example of the present invention. The main piston 4, which has two piston rods 6 in the embodiment example shown, separates the first hydraulic chamber 8 from the second hydraulic chamber 10. The two hydraulic chambers 8, 10 are connected by the fluid connection 12 where the valve 14 is located. In the embodiment example shown, the volume 18 is composed of two volumes 18. In the first volume is a first displaceable separating device 60 and in the second volume is a second displaceable separating device 62.

If the main piston 4 is displaced to the right in the representation shown, the first hydraulic chamber 8 becomes smaller and part of the fluid contained within is directed through the first partial line 24. The first separating device 60 is displaced to the right as a result. Part of the fluid situated to the right of the first separating device 60 within the corresponding volume 18 is displaced through the second partial line 26 into the second hydraulic chamber 10. Conversely, if the main piston 4 is displaced to the left, the second hydraulic chamber 10 becomes smaller and part of the fluid contained within is directed through the second partial line 26. As a result, the second displaceable separating device 62 is displaced to the right and part of the fluid there is pumped through the first partial line 24 into the first hydraulic chamber 8. The respective flow resistance can be set individually for both directions using the combinations of non-return and throttle valve upstream of the two volumes 18 and the spring elements contained in the volumes 18.

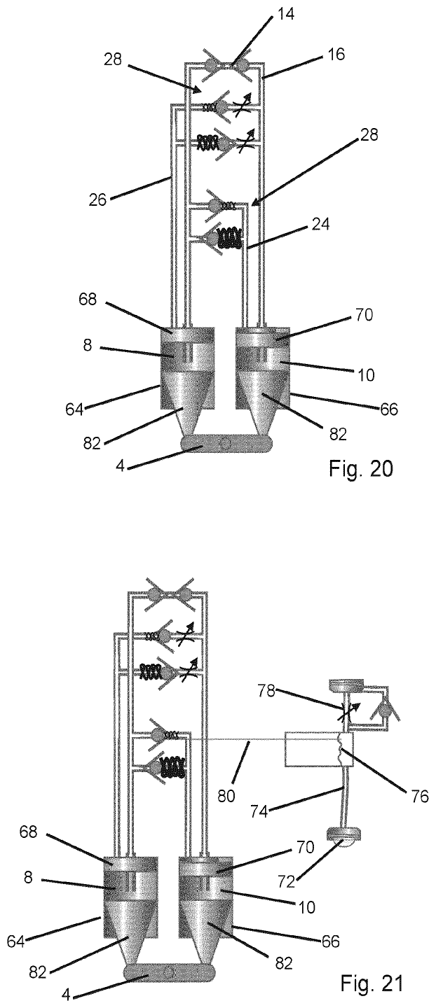

FIG. 20 shows a further switching arrangement of a hydraulic system for a joint according to an embodiment example of the present invention. In the representation shown, the first hydraulic chamber 8 and the second hydraulic chamber 10 are delimited at the bottom by the main piston 4. It comprises two single pistons 82, one of which projects into a first cylinder 64 and one into a second cylinder 66, thus delimiting the first hydraulic chamber 8 and the second hydraulic chamber 10. On the opposite side, the two hydraulic chambers 8, 10 are delimited by a first piston 68 and a second piston 70. The two chambers 8, 10 are connected to each other by the fluid connection 16, which can be opened and closed via the valve 14.

The second volume is located above the first piston 68, the former being arranged together with the first hydraulic chamber 8 in the first cylinder 64. In the situation shown in FIG. 20 it has no volume and is empty. The first volume is located above the second piston 70, the former being arranged together with the second hydraulic chamber 10 in the second cylinder 66. The first hydraulic chamber 8 is connected to the first partial volume via the first partial line 24. The second hydraulic chamber 10 is connected to the second partial volume via the second partial line 26.

A valve arrangement 28 is located in both the first partial line 24 and the second partial line 26. The valve 14 is opened in order to adjust the heel height of a prosthetic foot equipped with the hydraulic system. If the valve 14 is opened, the first joint part can be moved relative to the second joint part, causing fluid to be exchanged between the first hydraulic chamber 8 and the second hydraulic chamber 10.

The remaining individual valves of the two valve arrangements 28 determine the flow resistance in various situations. The upper valve of the valve arrangement 28 in the second partial line 26 in the representation is a non-return valve through which the fluid can flow from the second partial volume into the second hydraulic chamber 10 as occurs, for example, during dorsal flexion when the wearer is walking downhill. In the reverse direction, the fluid can only flow through said valve when the hydraulic pressure of the fluid is insufficient to compress the small spring, depicted to the left of the non-return valve, and thus close the valve. In this case, fluid can flow on this path from the second hydraulic chamber 10 into the second partial volume. This occurs, for example, when a relatively small heel load is applied for a long time, e.g. over several minutes.

The valve depicted below it is a non-return valve through which the fluid can flow in the opposite direction. This happens, for example, when the fast plantar flexion after heel strike is followed by a slow further plantar flexion, which can take place when walking downhill, for example.

The upper of the two valves of the valve arrangement 28 in the first partial line 24 is a non-return valve, through which the fluid can flow from the first partial volume into the first hydraulic chamber 8. This is practical, for example, when the wearer places the foot under the seating surface whilst sitting, causing a relatively slow dorsal flexion. The non-return valve shown below allows the fluid to take the opposite path. This occurs, for example, during dorsal flexion when going uphill.

FIG. 21 corresponds to the representation from FIG. 20, wherein an additional hydraulic element is provided, by way of which the upper valve of the valve arrangement 28 in the partial line 24 can be opened. The additional element has a first cushion 72, which is mechanically compressed when the two joint components reach a certain position relative to one another or according to another criterion. For example, the criterion may be the so-called “toe lift”, i.e. the moment at which the foot loses contact to the ground and moves into the swing phase of a step.

If the cushion 72 is compressed, fluid inside it flows through the line 74 and moves the membrane 76. The throttle valve 78 creates a flow resistance that causes the membrane 76 to move because the fluid builds up in the line 74. This preferably causes the non-return valve to open mechanically, for example via a tappet 80, and fluid can flow quickly and almost unobstructed from the first hydraulic chamber 8 into the first partial volume. This can be achieved, for example, by way of a package of springs or a single spring that is positioned within the second cylinder 66 between its upper delimitation and the second piston 70 and that exerts a downward force on the second piston 70. The second piston 70 is displaced downwards as a result, causing the second hydraulic chamber 70 to also be displaced downwards. The prosthetic foot is thus raised in the swing phase.

FIG. 22 depicts a hydraulic system similar to the one shown in FIG. 20 in a prosthetic foot. The prosthetic foot comprises the first joint part 34 and the second joint part 38. The main piston 4 is connected to the second joint part 38. In the first joint part 34 is the first cylinder 64, in which the first hydraulic chamber 8 is located, as well as the second cylinder 66, in which the second hydraulic chamber 10 is located. In the diagram shown, each of the two hydraulic chambers 8, 10 is delimited by a single cup-shaped piston 82, the single pistons 82 each constituting part of the main piston 4. At the opposite end, the first hydraulic chamber 8 is delimited by the first piston 68 and the second hydraulic chamber 10 by the second piston 70. The two pistons 68, 70 rest against the upper end of the cylinders 64, 66, so that the partial volumes situated above in the representation shown contain no hydraulic fluid.

If the first joint part 34 is moved relative to the second joint part 38, the hydraulic fluid contained in the hydraulic chambers is moved according to the operating principle of the hydraulic system from FIG. 20.

FIG. 23 shows a different embodiment of a prosthetic foot. It differs from the design from FIG. 22 in that there is no second piston. The second hydraulic chamber 10 is delimited below by the single cup-shaped piston 82 and above by the end of the second cylinder 66. Conversely, the first cylinder 64 contains the first piston 68, which is not arranged at the upper end of the first cylinder 64 in the representation shown. The first hydraulic chamber 8 below is likewise delimited by a single cup-shaped piston 82.

In the representations according to FIGS. 20 to 23, the first piston 68 and the second piston 70 each form a separating device. There are therefore two separating devices in FIGS. 20 to 22. Each of these separating devices separates a first partial volume from a second partial volume. There are therefore two first partial volumes and two second partial volumes. Each first partial volume is connected to the first hydraulic chamber via a first partial line and each second partial volume is connected to the second hydraulic chamber via a second partial line. There are therefore also two first partial lines and two second partial lines.

The first piston 68 separates the second partial volume, which is situated above the first piston 68 in the diagrams, from a first partial volume, located below the first piston 68. Said second partial volume is connected to the second hydraulic chamber 10 via the second partial line 26 shown. Along with the first partial line, said first partial volume constitutes part of the first hydraulic chamber 8. As in the other embodiments, the first hydraulic chamber 8 forms a first common volume with the first partial line and the first partial volume, even if the individual components of this first common volume are not shown or recognizable separately.

The second piston 70 separates the first partial volume, which is situated above the second piston 70 in the diagrams, from a second partial volume, located below the second piston 70. Said first partial volume is connected to the first hydraulic chamber 8 via the first partial line 24 shown. Along with the second partial line, said second partial volume constitutes part of the second hydraulic chamber 10. As in the other embodiments, the second hydraulic chamber 10 forms a second common volume with the second partial line and the second partial volume, even if the individual components of this second common volume are not shown or recognizable separately.

### REFERENCE LIST

2 cylinder
4 main piston
6 piston rod
8 first hydraulic chamber
10 second hydraulic chamber
12 fluid connection
14 valve
16 fluid line
18 volume
20 piston
22 end stop
24 first partial line
26 second partial line
28 valve arrangement
30 throttle valve
32 non-return valve
34 second joint part
36 swivel axis
38 first joint part
40 spring element
42 non-return valve
44 push button
46 lever
48 adjustment valve
50 disc spring
52 driver
54 relief valve
56 opening mechanism
58 membrane
60 first separating device
62 second separating device
64 first cylinder
66 second cylinder
68 first piston
70 second piston
72 first cushion
74 line
76 membrane
78 throttle valve
80 tappet
82 single piston

1. A joint for an orthopedic device, comprising;
a first joint part;
a second joint part arranged on the first joint part such that one or more of first joint part or second joint part is swivellable a swivel axis;
a hydraulic system comprising
a first hydraulic chamber,
a second hydraulic chamber connected to the first hydraulic chamber by at least one fluid connection, and
at least one valve configured to open and close the at least one fluid connection,
wherein the hydraulic system is arranged and configured such that hydraulic fluid flows from the first hydraulic chamber into the second hydraulic chamber or from the second hydraulic chamber into the first hydraulic chamber when the first joint part is swivelled relative to the second joint part,
wherein the hydraulic system has at least one volume which comprises a first partial volume and a second partial volume,
wherein the hydraulic system has at least one further fluid line which comprises a first partial line and a second partial line,
wherein the first partial volume is connected to the first hydraulic chamber via the first partial line and the second partial volume is connected to the second hydraulic chamber via the second partial line, and
wherein the first partial volume is separated from the second partial volume by a displaceable separating device.

2. The joint according to claim 1, wherein one or more of the first partial line and the second partial line comprises at least one throttle, wherein the at least one throttle is configured to adjust a flow resistance through the further fluid line.

3. The joint according to claim 1 further comprising at least one valve arrangement arranged in the first partial line and/or the second partial line, wherein the at least one valve arrangement is configured to adjust one or more of a flow resistance through the first line and a flow resistance through the second partial line,
wherein when the at least one valve arrangement comprises a first valve arrangement in the first partial line and a second valve arrangement in the second partial line the flow resistance through the first partial line and the flow resistance through the second partial line can be adjusted the same or differently for different flow directions.

4. The joint according to claim 1 wherein the separating device is displaceable in at least one direction in each case against a spring force applied by a spring element.

5. The joint according to claim 1 further comprising at least one end stop configured to limit movement of the separating device in at least one direction.

6. The joint according to claim 5, wherein the at least one end stop is adjustable so that a range of movement of the separating device is adjustable.

7. The joint according to claim 1 wherein the first hydraulic chamber and the second hydraulic chamber are separated by a main piston arranged and configured such that the main piston is moveable by swivelling the first joint part relative to the second joint part.

8. The joint according to claim 6 wherein one or more of the first partial line and the second partial line extend through the main piston.

9. The joint according to claim 1 wherein the first hydraulic chamber and the second partial volume and/or the second hydraulic chamber and the first partial volume are arranged in a common cylinder.

10. The joint according to claim 9, wherein the first hydraulic chamber and the second partial volume are separated from each other by a first piston and/or the second hydraulic chamber and the first partial volume are separated from each other by a second piston.

11. The joint according to claim 1 further comprising at least one closing valve configured to seal one or more of the first partial line and the second partial line.

12. The joint according to claim 11, wherein the at least one closing valve is designed such that it is opened when the at least one valve is closed and vice versa.

13. A method for setting a starting position of a first joint part relative to a second joint part of a joint according to claim 1, comprising:
positioning the displaceable separating device in a predetermined rest position,
opening the fluid connection by activating the at least one valve,
swivelling the first joint part relative to the second joint part (34) until the starting position is reached, and
closing the fluid connection.

14. The method according to claim 13, wherein the starting position corresponds to a predetermined joint angle between the first joint part and the second joint part.

15. The method according to claim 13 wherein the positioning step is performed by displacing the separating device within the volume until it reaches an end stop.

16. The method according to claim 13 wherein the positioning step is performed without torque acting about the swivel axis by application to the first joint part and/or the second joint part.

17. An orthopedic device with a joint according to claim 1 wherein the joint is a hip joint, an ankle joint, or a knee joint.

18. The joint according to claim 4 wherein the separating device is displaceable in two opposite directions.

19. The joint according to claim 5 wherein the at least one end stop limits movement of the separating device in two opposite directions, and wherein the at least one end stop comprises a damping element.

20. The method according to claim 15 wherein displacing the separating the device within the volume is performed by applying a torque acing about a swivel axis of one or more of the first joint part and the second joint part.
